(19) United States
(12) Patent Application Publication
(10) Pub. No.: US 2024/0033113 A1
(43) Pub. Date: Feb. 1, 2024

(54) ANTI-MIGRATION STENT

(71) Applicant: BOSTON SCIENTIFIC SCIMED, INC., Maple Grove, MN (US)

(72) Inventors: Rebecca Lenehan, Mayo (IE); Garrett Casserly, Galway (IE); Martin Murray, Loughrea (IE); Michael Walsh, Galway (IE); Paul E. Tierney, Athenry (IE)

(73) Assignee: BOSTON SCIENTIFIC SCIMED, INC., Maple Grove, MN (US)

(21) Appl. No.: 18/361,172

(22) Filed: Jul. 28, 2023

Related U.S. Application Data
(60) Provisional application No. 63/393,516, filed on Jul. 29, 2022.

(51) Int. Cl. A61F 2/94; A61F 2/86

(52) U.S. Cl. CPC A61F 2/94; A61F 2/86

(57) ABSTRACT

A stent includes an expandable tubular body formed of one or more interwoven wires, and including a plurality of anti-migration features each having a first end fixed to the tubular body and a second end extending radially outward from an outer surface of the tubular body. The anti-migration features may be formed of a closed loop of one or more of the interwoven wires extending from the outer surface of the tubular body. The closed loops may be formed at the first end, the second end and/or along a medial region of the tubular body. In some instances, the base of the loops may be a cross-over point of the wire(s) forming the closed loop. The wire(s) may be welded at the cross-over point.

### CROSS REFERENCE TO RELATED APPLICATIONS

This application claims the benefit of and priority to U.S. Provisional Patent Application Ser. No. 63/393,516 filed on Jul. 29, 2022, the disclosure of which is incorporated herein by reference.

### TECHNICAL FIELD

The disclosure pertains to medical devices and more particularly to implantable stents with anti-migration features, and methods for using such medical devices.

### BACKGROUND

A wide variety of medical devices have been developed for medical use including, for example, medical devices utilized in the treatment of bodily lumens. One type of intraluminal prosthesis used in the repair and/or treatment of diseases in various body lumens is a stent. A stent is a generally longitudinal tubular device formed of biocompatible material which is useful to open and support various lumens in the body. Stents may be used in various lumens in the body, such as in the vascular system, biliary tract, urogenital tract, gastrointestinal tract, esophageal tract, tracheal/bronchial tubes and bile duct, as well as in a variety of other lumens in the body. Of the known medical devices and methods, each has certain advantages and disadvantages. There is an ongoing need to provide alternative medical devices as well as alternative methods for manufacturing and using the medical devices.

### SUMMARY

This disclosure provides design, material, manufacturing method, and use alternatives for medical devices. An example stent includes a tubular body formed of interwoven wires, the tubular body having a first open end, an opposing second open end, and a central longitudinal axis extending therebetween, the tubular body moveable between a radially compressed state and a radially expanded state, and a plurality of anti-migration features each having a first end positioned at an outer surface of the tubular body and a second end extending radially outward from the outer surface of the tubular body, wherein each of the plurality of anti-migration features is defined by a closed loop of one or more of the interwoven wires with a base of the closed loop located at the outer surface of the tubular body.

Alternatively or additionally to the embodiment above, the base of the closed loop includes a cross-over point of the one or more interwoven wires forming the closed loop.

Alternatively or additionally to any of the embodiments above, the one or more interwoven wires are welded at the cross-over point.

Alternatively or additionally to any of the embodiments above, any pulling or squeezing force applied to any of the plurality of anti-migration features does not reduce an outer diameter of the tubular body or axially lengthen or shorten the tubular body.

Alternatively or additionally to any of the embodiments above, a first portion of the plurality of anti-migration features are coupled to the tubular body adjacent the first open end and extend towards the second open end at an acute angle relative to the outer surface of the tubular body.

Alternatively or additionally to any of the embodiments above, a second portion of the plurality of anti-migration features is coupled to the tubular body adjacent the second open end and extend towards the first open end at an acute angle.

Alternatively or additionally to any of the embodiments above, a first portion of the plurality of anti-migration features is coupled to a medial region of the tubular body and extend towards the first open end at an acute angle, and a second portion of the plurality of anti-migration features is coupled to the medial region of the tubular body and extend towards the second open end at an acute angle.

Alternatively or additionally to any of the embodiments above, the base of each anti-migration feature of the first portion and the base of each anti-migration feature of the second portion are circumferentially spaced apart at a single longitudinal location along the tubular body.

Alternatively or additionally to any of the embodiments above, the closed loops defining the plurality of anti-migration features are located at the first open end and extend radially outward from the tubular body.

Alternatively or additionally to any of the embodiments above, the stent further includes a plurality of elongated closed loops at the first open end extend substantially parallel to the central longitudinal axis.

Alternatively or additionally to any of the embodiments above, the plurality of elongate closed loops is interposed between adjacent ones of the closed loops defining the plurality of anti-migrations features.

Alternatively or additionally to any of the embodiments above, each closed loop is formed by a plurality of the interwoven wires, wherein terminal ends of the plurality of interwoven wires are welded around a periphery of the closed loop.

Alternatively or additionally to any of the embodiments above, each closed loop is formed by segments of four of the interwoven wires collectively defining a periphery of the closed loop.

Alternatively or additionally to any of the embodiments above, the base includes a cross-over point of first and second wires of the interwoven wires forming the closed loop.

Alternatively or additionally to any of the embodiments above, the first and second wires are welded together at the cross-over point.

Another example stent includes a tubular body formed of interwoven wires, the tubular body having a first open end, an opposing second open end, and a central longitudinal axis extending therebetween, the tubular body moveable between a radially compressed state and a radially expanded state, and a plurality of anti-migration features each having a first end welded to one or more cross-over points of the one or more interwoven wires forming the tubular body, and a second end extending radially outward from an outer surface of the tubular body.

Alternatively or additionally to the embodiment above, each of the plurality of anti-migration features is formed, at least in part, by a wire of the interwoven wires forming the tubular body.

Alternatively or additionally to any of the embodiments above, each of the plurality of anti-migration features is formed by a plurality of wires of the interwoven wires arranged in a closed loop, wherein terminal ends of the plurality of wires are welded around a periphery of the closed loop.

A further example stent includes a radially expandable tubular body formed of interwoven wires, the tubular body having a first open end, an opposing second open end, and a central longitudinal axis extending therebetween, the tubular body moveable between a radially compressed state and a radially expanded state, and a plurality of anti-migration features located at the first open end, each of the plurality of anti-migration features having a first end positioned at an outer surface of the tubular body and a second end extending radially outward from the outer surface of the tubular body, wherein each of the plurality of anti-migration features is formed by a plurality of wires of the interwoven wires arranged in a closed loop with terminal ends of the plurality of wires arranged around a periphery of the closed loop.

Alternatively or additionally to the embodiment above, the terminal end of the plurality of wire are welded around the periphery of the closed loop.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an example stent;

FIG. 1A is an exemplary view of an end region of the stent of FIG. 1;

FIGS. 1B and 1C are alternative end regions of the stent of FIG. 1;

FIGS. 2-8 are side views of additional example stents;

FIG. 9A is a partial side cross-sectional view of another example stent;

FIG. 9B is an end view of the stent of FIG. 9A;

FIG. 10 is a side view of another example stent;

FIG. 11A is a side cross-sectional view of a further example stent deployed between two body lumens; and

FIG. 11B is a perspective end view of the stent of FIG. 11A extending through tissue.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner. The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

Migration of stents may occur with self-expanding stents, such as fully covered stents. Current self-expanding stents as exemplified by those used in endoscopic applications may have features that promote anti-migration, such as flared or tapered regions. These mechanical changes have various degrees of success for reducing migration of the stent.

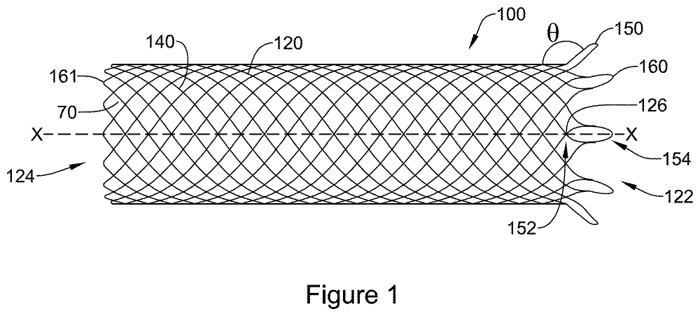

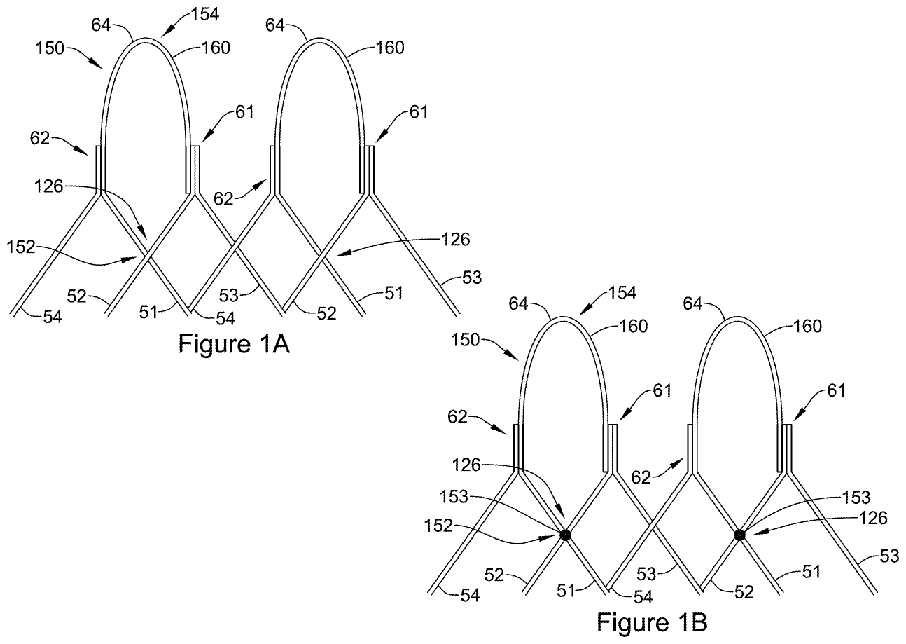

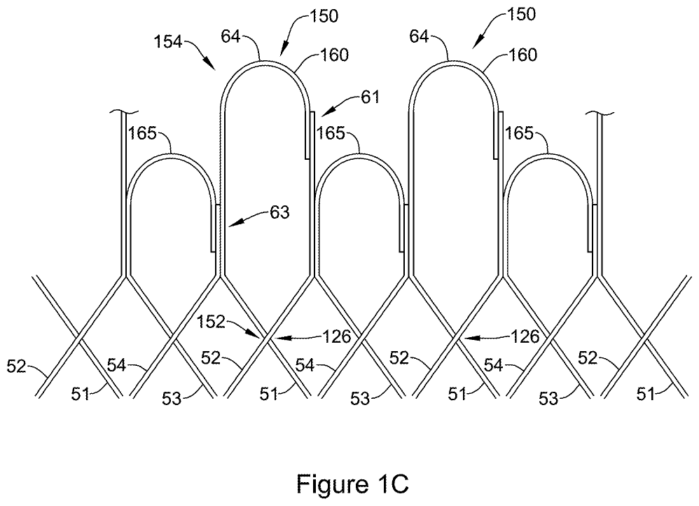

FIG. 1 illustrates an expandable stent 100 with a plurality of anti-migration features 150. The stent 100 includes an expandable tubular body 120 having a first open end 122 and an opposing second open end 124 and a central longitudinal axis X-X extending therebetween. The wall of the expandable stent 100 may define a lumen extending through the stent 100 along the central longitudinal axis X-X from the first open end 122 to the second open end 124. In some instances, the stent 100 may have a length from 30 mm to 200 mm and an outer diameter of from about 4 mm to about 28 mm, for example. These dimensions are only exemplary. Other lengths and/or diameters are also contemplated. The tubular body 120 may be expandable from a radially compressed delivery state to a radially expanded deployment state, as shown in FIG. 1. As is known in the art, the stent 100 may self-expand from the compressed delivery state to the expanded deployment state, or may be expanded by a balloon or other expansion device from the compressed delivery state to the expanded deployment state. The tubular body 120 may be defined by a stent wall having an interior surface and an exterior surface.

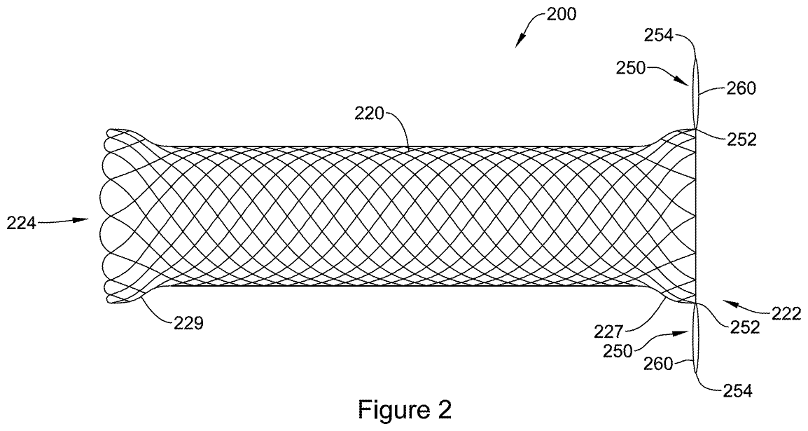

A stent 100 as described herein may have a tubular body 120 with a substantially constant diameter from the first open end 122 to the second open end 124, as shown in FIG. 1, or the stent 100 may have one or both end regions with a greater diameter than the middle region, such as shown in FIG. 2. Such a stent may be considered to have a first flared end region and/or a second flared end region, as desired.

The stent 100 may be formed of one or more, or a plurality of interwoven wires 140 forming the tubular body 120 of the stent 100. The interwoven wire(s) 140 may be knitted, braided, twisted, looped, or otherwise interwoven along the length of the tubular body 120. The tubular body 120 may include a series of closed loops at one or both of the opposing first and second open ends 122, 124, as shown in FIG. 1. As used herein, the term “closed loop” is intended to refer to a loop having an enclosed periphery in which the entire periphery of the loop is defined by one or more of the wires 140. In some embodiments one or both open ends 122, 124 may include elongated closed loops 160. As shown in FIG. 1, the closed loops at the first open end 122 are elongated closed loops 160, and the closed loops 161 at the second open end 124 are similar in size to the cells defined by the woven or braided wires 140 along the tubular body 120. In some instances, the terminal ends of each of the interwoven wires 140 may all be located at the first open end 122, such that the terminal ends of the interwoven wires 140 form the closed loops 160 at the first open end 122, while bent portions along a medial region of the interwoven wires 140 may form the closed loops 161 at the second open end 124. In other embodiments the terminal end of some of the interwoven wires 140 may be located at the second open end 124 while the terminal end of others of the interwoven wires 140 may be located at the first open end 122, if desired. In other embodiments, the tubular body 120 may be in the form of a mesh, laser cut from a tube, or laser cut from a sheet of material that is welded to form a tube.

The stent 100 may include a plurality of anti-migration features 150 extending radially outward from the outer surface of the tubular body 120. In some embodiments, one or more of the elongated closed loops 160 formed at one or both of the first open end 122 and the second open end 124 may define the anti-migration features 150. The anti-migration features 150 may be formed by the one or more, or plurality of interwoven wires 140 forming the tubular body 120. In this embodiment, the anti-migration features 150 may be formed with the same wires that are interwoven to form the body of the stent 100. In other embodiments, the anti-migration features 150 may be formed separately and then fixed to the tubular body 120 at, for example, wire cross-over points 126. In such instances, the anti-migration features 150 may be attached to the tubular body 120 by welding or adhesive bonding the base of the anti-migration features to the interwoven wires forming the body of the stent 100, for example.

As shown in FIGS. 1A, 1B, and 1C, the elongated closed loops 160 at the first open end 122 have been bent outward to form anti-migration features 150. Each of the anti-migration features 150 or closed loops 160 may have a first end 152 located at the wall of the tubular body 120 or otherwise coupled to the tubular body 120 and a second end 154 extending radially outward from an outer surface of the tubular body 120 to an apex of the anti-migration loop 160. As described herein, the direction that the anti-migration loop 160 extends is in the direction from the first, base end 152 located at the tubular body 120 to the second, free end 154 of the anti-migration loop 160. The anti-migration features 150 (e.g., the anti-migration loop 160) may have a length measured from the first end 152 to the second end 154. In some embodiments at least some of the anti-migration features 150 may have a length of at least one-third the outer diameter of the tubular body 120 measured at the widest point of the tubular body 120. In other embodiments, the length of the anti-migration features 150 may be at least one-half the outer diameter of the tubular body 120. In some instances, the first end 152, or base, of each anti-migration feature 150 (e.g., the anti-migration loop 160) may be secured or fixedly attached to the tubular body 120 such that any pulling or squeezing force applied in any direction to the anti-migration features 150 (e.g., the anti-migration loop 160) does not cause the anti-migration feature 150 (e.g., the anti-migration loop 160) to be increased or reduced in size and does not reduce or expand an outer diameter of the tubular body 120 or lengthen or shorten the tubular body 120. The size of each anti-migration feature 150 may thus be fixed in some instances. In some instances, the base or first end 152 of the closed loop 160 is formed by a first wire 51 crossing over/under a second wire 52 as the wires 51/52 extend from the tubular body 120 to begin forming the closed loop 160. In other words, in embodiments in which the anti-migration features 150 are formed from a plurality of wires 140 forming the interwoven tubular body 120, the first end 152 may be defined by a cross-over point 126 where two wires 140 extend from the interwoven tubular body 120 and cross over one another as the wires 140 form the anti-migration feature 150. Thus, the cross-over point 126 of the first and second wires 51/52 may define a portion of the enclosed periphery of the closed loop 160. In some instances, the first end 152, or base, of each anti-migration closed loop 160 of a plurality of anti-migration loops 160 may be arranged at a single circumferential row of cross-over points 126 at the first end 122 of the stent 100. In other words, the cross-over points of a plurality of anti-migration loops 160 at an end of the stent may be located circumferentially around the tubular body 120 at a single longitudinal location.

An enlarged view of the end region of the stent 100 of FIG. 1 is shown in FIG. 1A. As shown in FIG. 1A, each closed loop 160 forming an anti-migration feature 150 may be formed from a plurality of wires 140 extending from the interwoven tubular body 120 and secured (e.g., welded) to one another. For example, a first wire 51, extending along the interwoven tubular body 120 in a first helical direction may extend from the interwoven tubular body 120 and be bent around to form the apex 64 of the loop 160 at the free end 154 of the loop 160. A second wire 52, extending along the interwoven tubular body 120 in a second helical direction opposite the first helical direction, may cross over the first wire 52 at the base end 152 of the loop 160 and may have a terminal end joined to the terminal end of the first wire 51 along a perimeter of the loop 160 at a first fixation location 61. A third wire 53, which may extend along the interwoven tubular body 120 in the first helical direction parallel to the first wire 51, may have a terminal end joined to the terminal ends of the first wire 51 and/or the second wire 52 at the first fixation location 61. A fourth wire 54, which may extend along the interwoven tubular body 120 in the second helical direction parallel to the second wire 52, may have a terminal end joined to the first wire 51 at a second fixation location 62. The first and second fixation locations may be weld locations in some instances. For example, the terminal ends of the first, second, and third wires may be welded together at the first fixation location 61, and the terminal end of the fourth wire 54 may be welded to the first wire 51 at the second fixation location 62. The first and second fixation locations may be on opposite sides of the loop 160, for example. Thus, the periphery of each loop 160 may be formed of a portion of four individual wires 140 of the tubular body 120. Thus, in some instances, the stent 100 may include four times as many wires forming the tubular body 120 as loops 160 at the first end of the stent 100. In the embodiment of FIG. 1A, the first wire 51 may cross over the second wire 52 but not be secured to the second wire 52 at the cross-over point 126 at the base 152 of the loop 160. In another embodiment, the first wire 51 may cross over the second wire 52 at the cross-over point 126 at the base 152 of the loop 160 and be welded together at the cross-over point 126, as shown in FIG. 1B. With the wires 51/52 welded together at the cross-over point 126, deflection of the anti-migration features 150 may not reduce or increase the diameter or length of the tubular body 120, and as such would not function as retrieval elements.

The second end 154 of the closed loop 160 may be a free end forming the apex 64 of the closed loop 160. The second end 154 of at least some of the anti-migration features 150 may extend radially outward beyond the outermost extent of the outer surface of the tubular body 120. In some instances, the first end 152 of the anti-migration features 150 may include a single attachment point to the tubular body 120, while in other instances, the first end 152 of the anti-migration features 150 may include multiple attachment points to the tubular body 120. For example, an anti-migration feature 150 may be defined by a wire 140 or a plurality of wires 140 exiting and then re-entering the tubular body 120 at different spaced apart locations such that the first end 152 of the anti-migration feature 150 is defined by multiple wire cross-over points 126 and/or multiple weld locations. In some embodiments, the first end 152 of the anti-migration feature 150 that is coupled to the tubular body 120 may be defied at a single wire cross-over point 126 where two wires (e.g., a first wire 51 and a second wire 52) cross one another as they extend from the tubular body 120, as shown in FIGS. 1A and 1B. In some instances, a weld 153 may secure the first end 152 against enlargement of the anti-migration feature 150 at the cross-over point 126, as shown in FIG. 1B. Regardless of how many wire cross-over points 126 are involved in defining the anti-migration features 150, the wire cross-over points 126 may be welded such that any pulling or compression force applied to the anti-migration features 150 does not result in altering the diameter of or lengthening/shortening the tubular body 120. In such instances, the anti-migration features 150 do not function as retrieval elements to compress and/or lengthen the stent 100 for removal.

In another embodiment, shown in FIG. 1C, the first end 122 of the stent 100, may include a plurality of large closed loops 160 forming the anti-migration features 150 with a plurality of smaller closed loops 165 interposed between adjacent ones of the larger closed loops 160. Each closed loop 160 forming an anti-migration feature 150, as well as each smaller closed loop 165, may be formed from a plurality of wires 140 extending from the interwoven tubular body 120 and secured to one another. For example, a first wire 51, extending along the interwoven tubular body 120 in a first helical direction may extend from the interwoven tubular body 120 and be bent around to form the apex 64 of the large closed loop 160 at the free end 154 of the closed loop 160. A second wire 52, extending along the interwoven tubular body 120 in a second helical direction opposite the first helical direction, may cross over the first wire 52 at the base end 152 of the loop 160 and may have a terminal end joined to the terminal end of the first wire 51 along a perimeter of the loop 160 at a first fixation location 61. A third wire 53, which may extend along the interwoven tubular body 120 in the first helical direction parallel to the first wire 51, may be bent around to form an apex of the smaller closed loop 165. A fourth wire 54, which may extend along the interwoven tubular body 120 in the second helical direction parallel to the second wire 52, may have a terminal end joined to the third wire 53 at a second fixation location 63. In some instances, the larger loops 160 alternate with the smaller loops 165 around the circumference of the end 122 of the tubular body 120 of the stent 100. In some instances, the smaller loops 165 may be juxtaposed with the larger closed loops 160 without the perimeter of the smaller closed loops 165 secured to the perimeter of the lager closed loops 160. In other words, the periphery of the larger loops 160 may be free from securement (e.g., welding) to the periphery of the smaller loops 165 such that the larger loops 160 may be freely deflectable relative to the smaller loops 165. The larger closed loops 160 may be bent radially outward relative to the smaller loops 165. For example, the smaller loops 165 may extend longitudinally substantially parallel to the wall of the tubular body 120, whereas the large loops 160 may extend radially outward at an oblique (i.e., acute or obtuse) or perpendicular angle to the smaller loops 165.

The anti-migration features 150 (some example of which have been illustrated in FIGS. 1A-1C) may extend outward from the outer surface of the tubular body 120 at an angle (such as an oblique or perpendicular angle) relative to the central longitudinal axis and/or outer surface of the tubular body 120. In some instances, the angle may be an obtuse angle, as shown in FIG. 1 in which the anti-migration features 150 extend toward the first open end 122. In other instances, the angle may be an acute angle in which the anti-migration features 150 are bent back toward the second open end 124, if desired. In yet other instances, the angle may be a perpendicular angle. In some instances, the angle θ may be between about degrees to about 160 degrees, between about 100 degrees to about 160 degrees, between about 100 degrees to about 140 degrees, between about 90 degrees to about 120 degrees, between about 20 degrees to about 90 degrees, between about 30 degrees to about 80 degrees, between about 20 degrees to about 45 degrees, etc. The second end 154 of at least some of the anti-migration features 150 extends radially outward beyond the outermost extent of the surface of tubular body 120. The plurality of anti-migration features 150 may be biased in the extended, angled position relative to the tubular body 120 when unconstrained and/or the stent is deployed to the expanded configuration.

In some embodiments the stent 100 may include a covering 70 (see FIG. 1) disposed over at least a portion of the tubular body 120 of the stent 100. For example, the covering 70 may fully cover the entire length of the tubular body 120 of the stent 100, forming a fully covered stent in which all of the interstices or closed cells defined in the interwoven pattern (e.g., braided pattern) of the tubular body 120 are covered with the covering 70 to prevent tissue in-growth and/or fluid leakage into the lumen of the tubular body 120. In other examples, the covering 70 may cover only a portion of the length of the tubular body 20 of the stent 100 forming a partially covered stent in which a portion of the interstices or closed cells defined in the interwoven pattern (e.g., braided pattern) remain uncovered, allowing tissue in-growth. In some instances, the anti-migration features 150 may be covered by the covering 70, thus the entire stent 100, including both the entire tubular body 120 and the anti-migration features 150 and closed loops 60 may be covered by the covering 70. In some instances, the stent 100 may be dipped into a solution of silicone or other polymer to form the covering 70 or the stent 100 may be spray coated with a silicone or other polymer to form the covering 70. In other instances, a polymer sheet or tube may be placed around the tubular body 120 and/or within the tubular body 120 to form the covering 70. The covering 70 may be disposed on external or internal surfaces of the tubular body 120, or on both the internal and external surfaces of the tubular body 120, thereby embedding the tubular body 120 of the stent 100 in the polymeric material. The coating or covering may be a polymer covering, such as a polytetrafluoroethylene (PTFE) or silicone covering, however other coverings, particularly elastomeric polymers, may be used. Non-limiting examples of useful polymeric materials include polyesters, polypropylenes, polyethylenes, polyurethanes, polynaphthalenes, polytetrafluoroethylenes, expanded polytetrafluoroethylene, silicone, and combinations and copolymers thereof.

In another embodiment, a stent 200, similar to the stent 100, may have a plurality of anti-migration features 250 formed of closed loops 260 extending radially outward from the outer surface of the tubular body 220 at an angle of about 90 degrees, as shown in FIG. 2. The anti-migration features 250 may be configured as a closed loop formed of one or more, or a plurality of wires forming the interwoven tubular body 200, similar to the anti-migration features 150 described above. The plurality of anti-migration features 250 may be biased in the extended, angled position relative to the tubular body 220 when unconstrained and/or the stent is deployed to the expanded configuration. The tubular body 220 may include a first outwardly flared region 227 at the first open end 222 and/or a second outwardly flared region 229 at the second open end 224. The first and/or second outwardly flared regions 227, 229 may have an outer diameter larger than an outer diameter of a remainder of the tubular body 220. The plurality of anti-migration features 250 may extend from the first and/or second outwardly flared region 227, 229. While only two anti-migration features 250 (e.g., closed loops) are viewable in the side view shown in FIG. 2, it will be understood that additional anti-migration features 250 (e.g., closed loops) may extend around the circumference of the first open end 222, similar to the arrangement shown in FIG. 1. In some instances, the first end 252, or base, of each anti-migration closed loop 260 of a plurality of anti-migration loops 260 may be arranged at a single circumferential row of cross-over points of wires forming the tubular body 220 at the first end 222 of the stent 200. In other words, the cross-over points of a plurality of anti-migration loops 260 at an end of the stent may be located circumferentially around the tubular body 220 at a single longitudinal location.

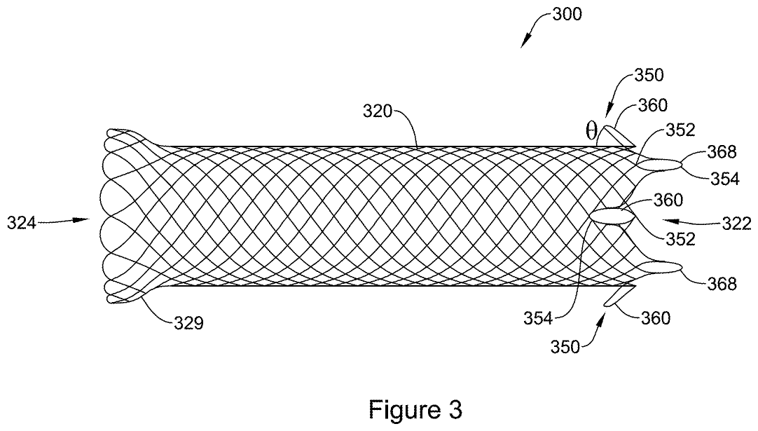

FIG. 3 illustrates a further embodiment of a stent 300 in which the plurality of anti-migration features 350 formed as closed loops 360 alternates with elongated closed loops 368 extending parallel to the longitudinal axis of the tubular body 320, where the anti-migration features 350 are formed at the first open end 322 of the tubular body 320. The closed loops 360, as well as the closed loops 368 may be formed similar to the closed loops 160 described above. It will be understood that the anti-migration features 350 and longitudinal elongated closed loops 368 may be in any arrangement, such as every second, third, fourth, or fifth loop being an anti-migration feature 350 and the remaining loops being longitudinal elongated closed loops 368. In some instances, each anti-migration loop 360 may be positioned circumferentially between adjacent ones of the longitudinal elongated closed loops 368. Additionally, the anti-migration features 350 and longitudinal elongated closed loops 368 may form an irregular pattern around the open end of the stent 300. The anti-migration features 350 may extend from the first open end 322, at an angle, (such as an oblique or perpendicular angle) relative to the central longitudinal axis and/or outer surface of the tubular body 320 of the stent 100. As shown in FIG. 3, in some instances, the closed loops 360 defining the anti-migration features 350 may extend toward the opposite end 324 of the stent 300 than the longitudinal elongated loops 368. In other instances, the closed loops 360 defining the anti-migration features 350 may extend toward the same end 322 of the stent 300 as the longitudinal elongated loops 368. In some instances, the anti-migration features 350 may extend about 20 degrees to about 60 degrees relative to the outer surface of the tubular body 320. In some instances, the angle θ may be between about 10 degrees to about 160 degrees, between about 100 degrees to about 160 degrees, between about 100 degrees to about 140 degrees, between about 90 degrees to about 120 degrees, between about 20 degrees to about 90 degrees, between about 30 degrees to about 80 degrees, between about degrees to about 45 degrees, etc. The plurality of anti-migration features 350 may be biased in the extended, angled position when unconstrained and/or the stent is deployed to the expanded configuration.

In some instances, the first end 352, or base, of each anti-migration closed loop 360 and each longitudinal elongated loop 368 at the first end 322 of the stent 300 may be arranged at a single circumferential row of cross-over points of wires forming the tubular body 320 at the first end 322 of the stent 300. In other words, the cross-over points of a plurality of anti-migration loops 360 and longitudinally extending loops 368 at an end of the stent may be located circumferentially around the tubular body 320 at a single longitudinal location. The second, free end 354 of the anti-migration loops 360 may extend in a first longitudinal direction from the circumferential row of base ends 322 while the second, free end 354 of the longitudinally extending loops 368 may extend in a second, opposite longitudinal direction from the circumferential row of base ends 322.

In the illustrated embodiment, the first open end 322 is substantially cylindrical with an outer diameter remaining constant from the first open end 322 to a second flared region 329 adjacent the second open end 324. Alternatively, the entire tubular body 320 may be cylindrical with a constant outer diameter, similar to the stent 100 shown in FIG. 1A.

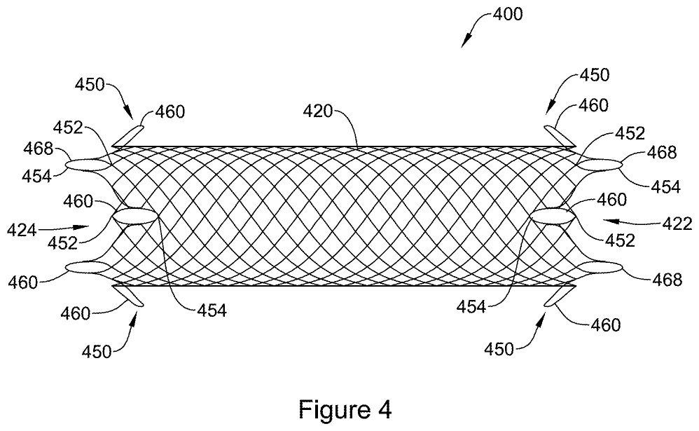

FIGS. 4-8 illustrate stents 400, 500, 600, 700, 800 with various arrangements of anti-migration features 450, 550, 650, 750, 850. The stent 400 shown in FIG. 4 includes a first portion of anti-migration features 450 (formed as closed loops 460) coupled to the stent 400 adjacent the first open end 422 and extending towards the opposite second open end 424 at an acute angle relative to the outer surface of the tubular body 420. A second portion of anti-migration features 450 (formed as closed loops 460) are coupled to the tubular body 420 adjacent the second open end 424 and extend towards the first open end 422 at an acute angle relative to the outer surface of the tubular body 420. Each of the closed loops 460 may be formed of one or more, or a plurality of wires forming the interwoven structure of the tubular body 420. In the illustrated embodiment, the first and second portions of anti-migration features 450 extend at an angle of about 20-30 degrees relative to the central longitudinal axis or outer surface of the tubular body 420, towards either the first or second open end 422, 424. However, in other instances, the anti-migration features 450 may extend at any desired angle, as discussed above. The plurality of anti-migration features 450 may be biased in the extended, angled position when unconstrained and/or the stent is deployed to the expanded configuration. In some embodiments, the tubular body 420 may include one or more elongated closed loops 468 extending from either or both of the first open end 422 and the second open end 424. These elongated closed loops 468 may extend parallel to a central longitudinal axis extending through the tubular body 420. In other embodiments, the elongated closed loops 468 may extend at an angle to the longitudinal axis different from the angle of the closed loops 460 and thus form additional anti-migration features. As shown in FIG. 4, the anti-migration closed loops 460 at the first end 422 of the stent 400 may extend toward the second end 424 while the longitudinal elongated closed loops 468 at the first end 422 extend toward the first end 422, and thus extend in a generally opposite direction as the anti-migration closed loops 460 at the first end 422. Additionally, the anti-migration closed loops 460 at the second end 424 of the stent 400 may extend toward the first end 422 while the longitudinal elongated closed loops 468 at the second end 424 extend toward the second end 424, and thus extend in a generally opposite direction as the anti-migration closed loops 460 at the second end 424.

In some instances, the first end 452, or base, of each anti-migration closed loop 460 and each longitudinal elongated loop 468 at the first end 422 and/or second end 424 of the stent 400 may be arranged at a single circumferential row of cross-over points of wires forming the tubular body 420 at the first end 422 of the stent 400 or the second end 424 of the stent 400, respectively. In other words, the cross-over points of a plurality of anti-migration loops 460 and longitudinally extending loops 468 at an end of the stent may be located circumferentially around the tubular body 420 at a single longitudinal location. The second, free end 454 of the anti-migration loops 460 may extend in a first longitudinal direction from the circumferential row of base ends 422 while the second, free end 454 of the longitudinally extending loops 468 may extend in a second, opposite longitudinal direction from the circumferential row of base ends 422.

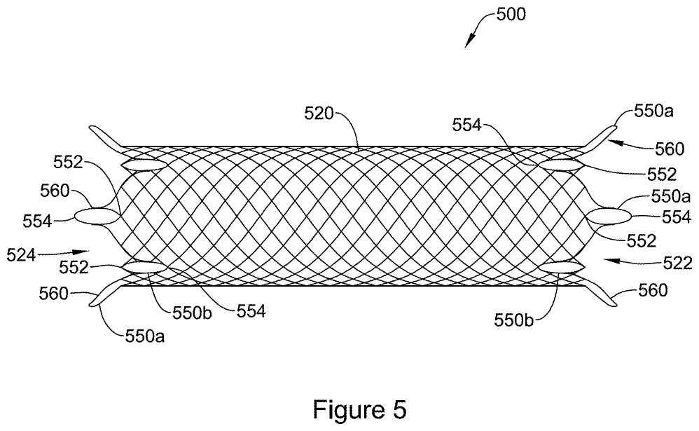

The stent 500 shown in FIG. 5 includes a first open end 522, a second open end 524, and a plurality of anti-migration features 550 extending at various angles at both open ends. In some instances, the first end 522 and/or the second end 524 may be a flared end having an outer diameter greater than the outer diameter of a medial region of the tubular body 520. As shown in FIG. 5, a first portion of the anti-migration features adjacent the first open end 522 may include anti-migration features 550a (e.g., closed loops 560) extending in a first longitudinal direction from the base 552 of the closed loops 560 and anti-migration features 550b (e.g., closed loops 560) extending in a second, opposite longitudinal direction from the base 552 of the closed loops 560. In some instances, the anti-migration features 550a extending away from a medial region of the stent 500 may extend at an angle of greater than degrees (e.g., between 100 degrees and 130 degrees) relative to the outer surface of the tubular body 520, and the anti-migration features 550b extending toward the medial region of the stent 500 may extend at an angle of less than 90 degrees (e.g., between 20 degrees and 85 degrees) relative to the outer surface of the tubular body 520. The anti-migration features 550 (i.e., the closed loops 560) may be formed similar to the other anti-migration features described herein. The plurality of anti-migration features 550 may be biased in the extended, angled position when unconstrained and/or the stent is deployed to the expanded configuration.

In some instances, the first end 552, or base, of each anti-migration closed loop 560 and each longitudinal elongated loop 568 at the first end 522 and/or second end 524 of the stent 500 may be arranged at a single circumferential row of cross-over points of wires forming the tubular body 520 at the first end 522 of the stent 500 or the second end 524 of the stent 500, respectively. In other words, the cross-over points of a plurality of anti-migration loops 560 and longitudinally extending loops 568 at an end of the stent may be located circumferentially around the tubular body 520 at a single longitudinal location. The second, free end 554 of the anti-migration loops 560 may extend in a first longitudinal direction from the circumferential row of base ends 522 while the second, free end 554 of the longitudinally extending loops 568 may extend in a second, opposite longitudinal direction from the circumferential row of base ends 522.

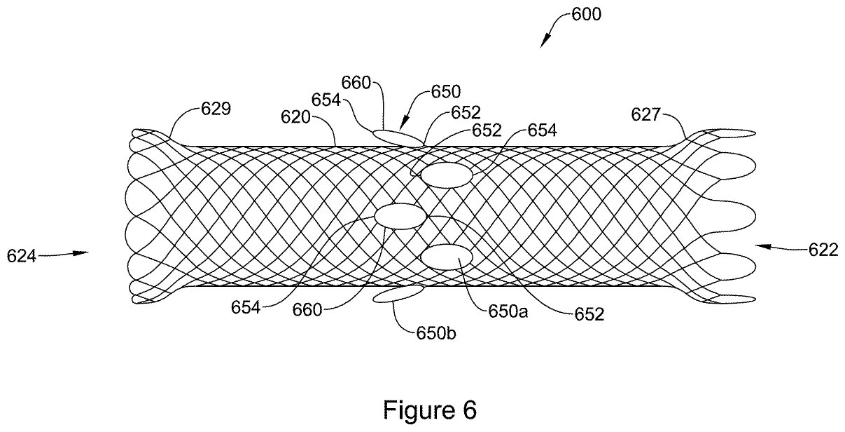

The stent 600 shown in FIG. 6 includes a first flared end 627 adjacent the first open end 622 and a second fared end 629 adjacent the second open end 624. A plurality of elongated loops, forming apices at the first open end 622 extending substantially parallel to a longitudinal axis of the tubular body 620 at the first open end 622. The stent 600 further includes a plurality of anti-migration features 650 in a medial region of the tubular body 620. The anti-migration features 650 may be formed as closed loops 660, similar to the other anti-migration features described herein. The anti-migration features 650 may include a first portion of anti-migration features 650a extending towards the first open end 622 and a second portion of anti-migration features 650b extending towards the second open end 624. As shown, the first and second portions of anti-migration features 650a, 650b alternate around a circumference of the tubular body 620. Each of the anti-migration features 650a, 650b may extend at an acute angle (such as between 10 degrees and 80 degrees) relative to the outer surface of the tubular body 620. In some embodiments, the anti-migration features 650a, 650b may each extend at different angles. The plurality of anti-migration features 650a, 650b may be biased in the extended, angled position when unconstrained and/or the stent is deployed to the expanded configuration.

In some instances, the first end 652, or base, of each anti-migration feature 650a (e.g., closed loop 660) extending toward the first end 622 and each anti-migration feature 650b (e.g., closed loop 660) extending toward the second end 624 may be arranged at a single circumferential row of cross-over points of wires forming the tubular body 620. In other words, the cross-over points of a plurality of anti-migration loops 660 extending in both longitudinal directions may be located circumferentially around the tubular body 620 at a single longitudinal location. The second, free end 654 of the anti-migration features 650a may extend in a first longitudinal direction from the circumferential row of base ends 622 toward the first end 622 while the second, free end 654 of the anti-migration features 650b may extend in a second, opposite longitudinal direction from the circumferential row of base ends 622 toward the second end 624.

The anti-migration features 650a, 650b may be formed by a radially extending loop in a wire forming the tubular body 620, where the loop extends radially outward from the outer surface of the tubular body 620. The wire loop may be a closed loop in which the wire crosses over itself at the base of the loop located at the tubular body 620 before entering the interwoven structure forming the tubular body 620. In some instances, the base of the loop (e.g., the cross-over point) may be welded such that the loop forming the anti-migration feature 650a, 650b cannot be enlarged or reduced in size. In other embodiments, the anti-migration features 650a, 650b may be formed by a wire loop formed separately and attached, such as by welding, to the tubular body 620 at a crossover point such that pulling or squeezing force on loop does not reduce the outer diameter or change the length of tubular body 620. In some instances, the wire forming the wire loop may not cross over itself at the base of the wire loop, but rather two segments of the wire may enter the interwoven structure forming the tubular body 620 at spaced apart locations. In some instances, the two wire segments may be welded to additional wires forming the tubular body 620 at the spaced apart locations in which the wire segments enter the interwoven structure forming the tubular body 620.

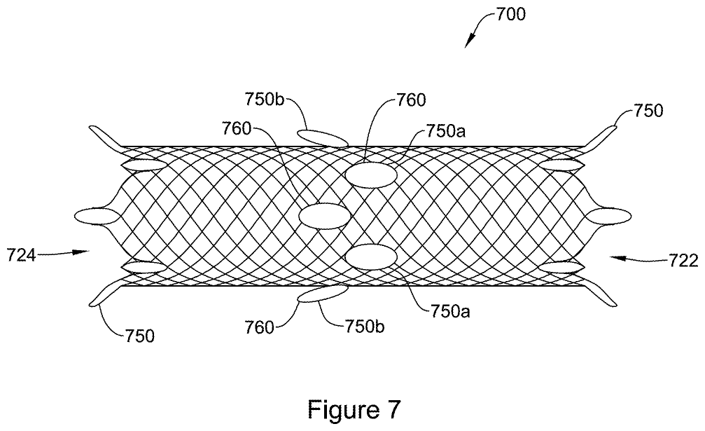

The stent 700 illustrated in FIG. 7 has a combination of the features of the stents 500, 600 shown in FIGS. 5 and 6, with a first portion of anti-migration features 750 adjacent the first open end 722, a second portion of anti-migration features 750 adjacent the second open end 724, and a third portion of anti-migration features 750 in a medial region of the stent 700. The discussion above, is applicable to the embodiment of FIG. 7. The anti-migration features may be formed of closed loops 760, similar to the other closed loop configurations described herein. The closed loops 760 in the medial region may include a first portion of anti-migration features 750a extending in a first longitudinal direction and a second portion of anti-migration features 750b extending in a second, opposite longitudinal direction. Similarly, the closed loops 760 at the first open end 722 may include a first portion extending in a first longitudinal direction and a second portion extending in a second, opposite longitudinal direction and/or the closed loops 760 at the second open end 724 may include a first portion extending in a first longitudinal direction and a second portion extending in a second, opposite longitudinal direction. In each of the first, second, and third portions, the anti-migration features 750 may extend at any desired oblique (e.g., acute or obtuse) or perpendicular angle to the central longitudinal axis or outer surface of the stent 700. For example, in some instances, the closed loops 760 may extend at an angle of 20 degrees to 120 degrees relative to the outer surface of the stent, towards either the first open end 722 or the second open end 724. The plurality of anti-migration features 750 may be biased in the extended, angled position when unconstrained and/or the stent is deployed to the expanded configuration. In some instances, the first and second portions of anti-migration features may be disposed on a first flared end region at the first end 722 and a second flared end region at the second end 724, respectively. Each of the anti-migration features 750 may extend at the same or a different angle.

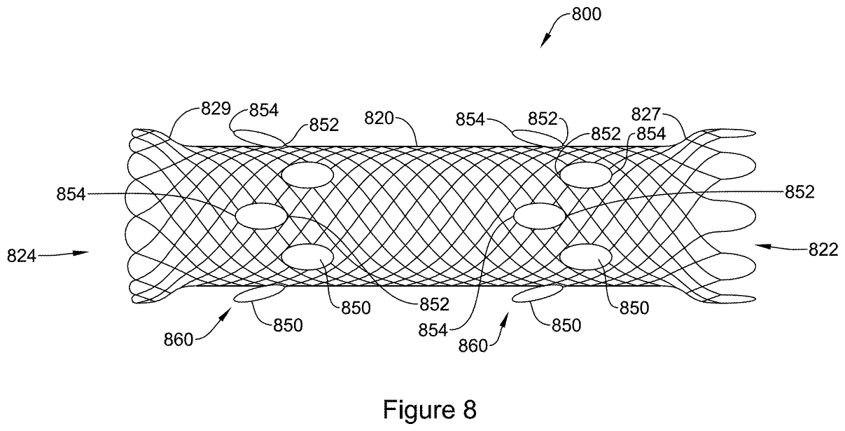

FIG. 8 illustrates a stent 800 having a first open end 822 with a first flared end region 827 and a second open end 824 with a second flared end region 829, each devoid of any anti-migration features. The first open end 822 and/or the second open end 824 may include one or more elongated loops, forming apices at the first open end 822 extending substantially parallel to a longitudinal axis of the stent 800. The stent 800 further includes a plurality of anti-migration features 850 may be disposed on a medial region of the tubular body 820 between the first and second open ends 822, 824. The anti-migration features 850 may be formed as closed loops 860, similar to the other anti-migration features described herein. The anti-migration features 850 may be present in a plurality of separate sets spaced apart longitudinally from one another, where each set includes a first portion of anti-migration features 850 (e.g., closed loops 860) extending toward the first open end 822 and a second portion of anti-migration features 850 (e.g., closed loops 860) extending toward the second open end 824. The anti-migration features 850 may alternate direction as shown in FIG. 8. The anti-migration features 850 may extend at any oblique (e.g., acute or obtuse) or perpendicular angle to the central longitudinal axis or outer surface of the stent 800 (such as at an angle of 20 degrees to 120 degrees relative to the outer surface of the stent), towards either the first open end 822 or the second open end 824. Each of the anti-migration features 850 may extend at the same or a different angle. The plurality of anti-migration features 850 may be biased in the extended, angled position when unconstrained and/or the stent is deployed to the expanded configuration.

In some instances, regarding a first set of anti-migration features 850 at a first location along the medial region of the stent 800, the first end 852, or base, of each anti-migration feature 850 (e.g., closed loop 860) extending toward the first end 822 and each anti-migration feature 850 (e.g., closed loop 860) extending toward the second end 824 may be arranged at a single circumferential row of cross-over points of wires forming the tubular body 820. In other words, the cross-over points of a plurality of anti-migration loops 860 extending in both longitudinal directions may be located circumferentially around the tubular body 820 at a first longitudinal location. The second, free end 854 of the first portion of the anti-migration features 850 may extend in a first longitudinal direction from the circumferential row of base ends 822 toward the first end 822 while the second, free end 854 of a second portion of the anti-migration features 850 may extend in a second, opposite longitudinal direction from the circumferential row of base ends 822 toward the second end 824.

The stent 800 may include a second set of anti-migration features 850 located at a second location along the medial region of the stent 800 spaced longitudinally away from the first set of anti-migration features 850. Regarding the second set of anti-migration features at the second location along the medial region of the stent 800, the first end 852, or base, of each anti-migration feature 850 (e.g., closed loop 860) extending toward the first end 822 and each anti-migration feature 850 (e.g., closed loop 860) extending toward the second end 824 may be arranged at a single circumferential row of cross-over points of wires forming the tubular body 820. In other words, the cross-over points of a plurality of anti-migration loops 860 extending in both longitudinal directions may be located circumferentially around the tubular body 820 at a first longitudinal location. The second, free end 854 of the first portion of the anti-migration features 850 may extend in a first longitudinal direction from the circumferential row of base ends 822 toward the first end 822 while the second, free end 854 of a second portion of the anti-migration features 850 may extend in a second, opposite longitudinal direction from the circumferential row of base ends 822 toward the second end 824.

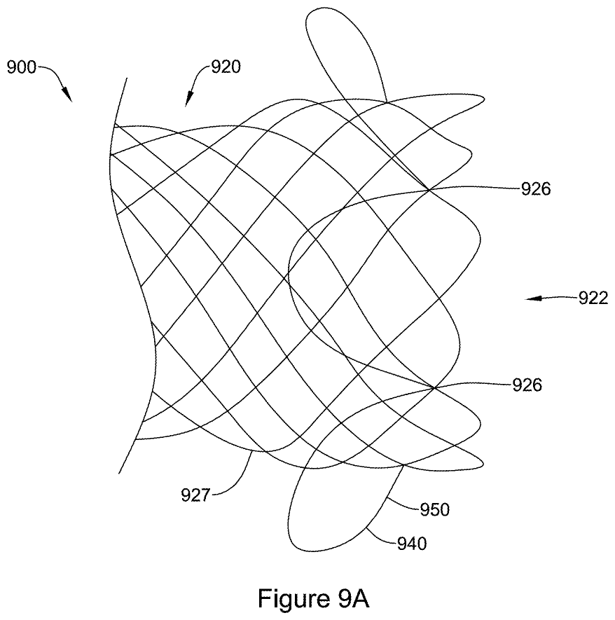

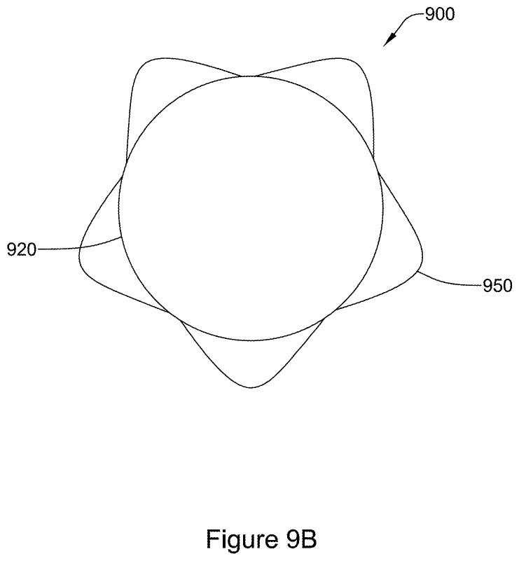

FIGS. 9A and 9B illustrate a portion of a stent 900 in which a plurality of anti-migration features 950 extend from a first open end 922 of the tubular body 920 of the stent 900. Each of the anti-migration features 950 may be defined by a looped portion of one the wires 940 extending between two cross-over points 926 as the wire 940 extends outward from the tubular body 920 of the stent 900. Thus, the anti-migration features 950 may be formed by a wire 940 extending between two circumferentially spaced apart cross-over pints 926, as shown in FIG. 9A. The wire 940 may be welded to additional wires forming the interwoven structure of the tubular body 920 at the two cross-over points 926 to prevent any pulling or squeezing force applied to the anti-migration feature 950 from reducing the outer diameter of or lengthening the stent 900. The anti-migration features 950 may extend radially outward from the outer surface of the tubular body 920 at any desired angle, such as an oblique (e.g., acute or obtuse) or perpendicular angle relative to the central longitudinal axis and/or outer surface of the tubular body 920. In some instances, the angle may be an obtuse angle in which the anti-migration features 950 extend toward the first open end 922. In other instances, the angle may be an acute angle in which the anti-migration features 950 are bent back toward the opposite, second open end of the stent 900 (note shown), if desired. In yet other instances, the angle may be a perpendicular angle. In some instances, the angle may be between about 10 degrees to about 160 degrees, between about 100 degrees to about 160 degrees, between about 100 degrees to about 140 degrees, between about 90 degrees to about 120 degrees, between about 20 degrees to about 90 degrees, between about 30 degrees to about 80 degrees, between about 20 degrees to about 45 degrees, etc. The anti-migration features 950 may form a petal structure when viewed from the end, as shown in FIG. 9B. The plurality of anti-migration features 950 may be biased in the extended, angled position relative to the tubular body 920 when unconstrained and/or the stent is deployed to the expanded configuration. The stent 900 may include a first flared region 927 adjacent the first open end 922, if desired. As shown in FIG. 9B, the first open end 922 may include a plurality of anti-migration features 950 arranged around the circumference of the tubular body 920 and extending radially outward therefrom.

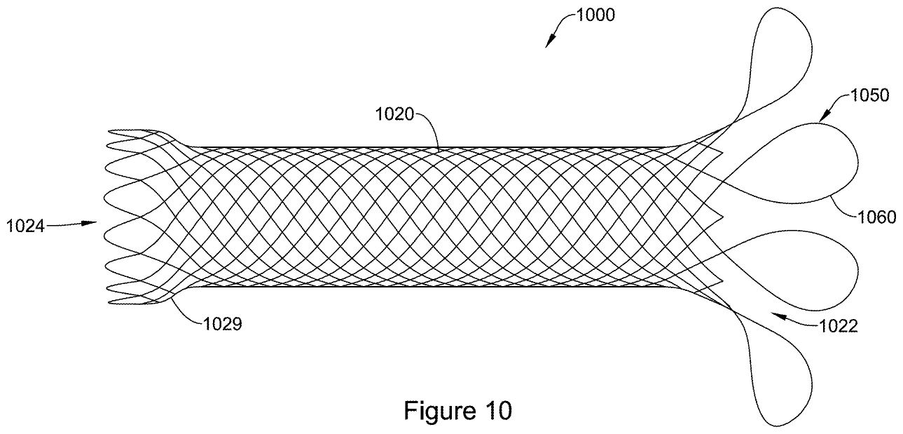

FIG. 10 illustrates a stent 1000 with an alternative anti-migration structure. In this embodiment, all of the closed loops 1060 at the first open end 1022 of the stent 1000 are greatly enlarged closed loops 1060 that define anti-migration features 1050. In some instances, the enlarged closed loops 1060 may be formed of a looped portion of a single wire crossing over itself at a cross-over point at the base of the closed loop 1060. In some embodiments, the enlarged closed loops 1060 may each have an outermost diameter of at least 2 times, at least 3 times, or at least 4 times the outer diameter of the tubular body 1020 of the stent 1000. In some instances, the enlarged closed loops 1060 may have a length at least one-half or more of the outer diameter of the tubular body 1020 forming the stent 1000 or a length equal to or greater than the outer diameter of the tubular body 1020 of the stent 1000. The enlarged closed loops 1060 may be oval or polygonal such as defining octagons. The enlarged closed loops 1060 may extend from the first open end 1022 at any desired angle relative to the central longitudinal axis and/or outer wall of the tubular body 1020. The plurality of anti-migration features 1050 may be biased in the extended, angled position when unconstrained and/or the stent is deployed to the expanded configuration.

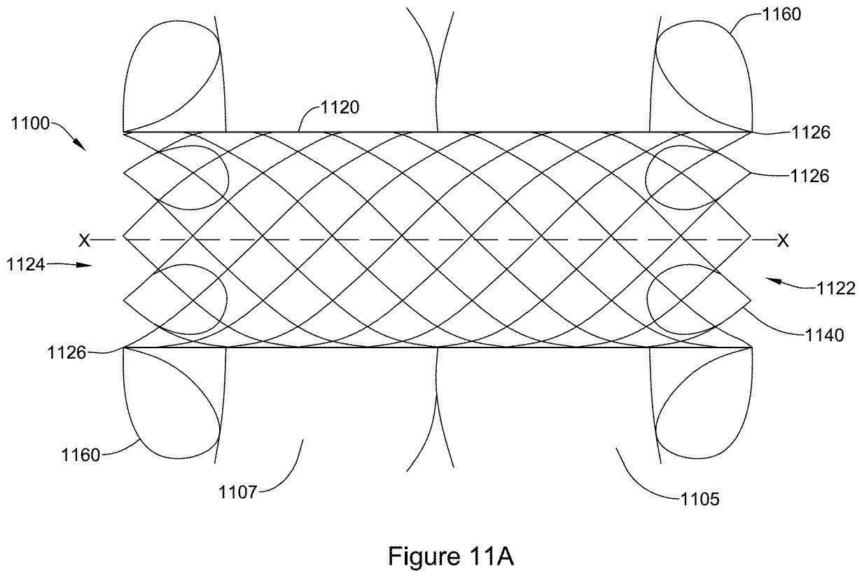

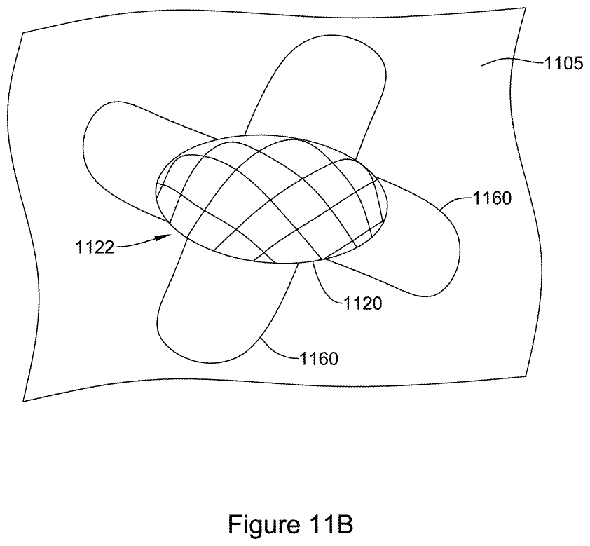

A further embodiment of the stent 1100 may have a plurality of enlarged closed loops 1160 extending from both the first open end 1122 and the second open end 1124, as illustrated in FIG. 11A. The enlarged closed loops 1160 may extend radially outward from the tubular body 1120 forming the stent 1100 at any desired angle, such as at an angle of about 45 degrees to about 90 degrees from a longitudinal axis X-X extending through the stent 1100. In some embodiments, each end of the enlarged closed loop 1160 may extend from a cross-over point 1126. The enlarged closed loops 1160 may be formed from the wires 1140 that form the tubular body 1120. In other embodiments, the enlarged closed loops 1160 may be formed separately and fixed to the tubular body 1120. Regardless of whether the enlarged closed loops 1160 are formed from the wires 1140 forming the tubular body 1120 or are formed separately and fixed to the tubular body 1120, the cross-over points 1126 from which the enlarged closed loops extend may be welded. This prevents any pulling or squeezing force applied to the enlarged closed loops 1160 from reducing the diameter of or elongating the tubular body 1120. As such, the enlarged closed loops 1160 do not form a retrieval or removal structure. The enlarged closed loops 1160 may be biased in the extended, angled position when unconstrained and/or the stent is deployed to the expanded configuration.

The stent 1100 may be used as a conduit establishing fluid communication between adjacent body lumens. For example, the stent 1100 may be used as a drainage stent, fistula, anastomosis, etc. The enlarged closed loops 1160 may be configured to engage and hold two adjacent body lumens 1105, 1107 in place for fluid to flow therebetween, as illustrated in FIGS. 11A and 11B. In one example, the stent 1100 may be used to drain bile and/or gallstones from the gallbladder to the duodenum. In another example, the stent 1100 may be used in an endoscopic procedure such as a gastrojejunostomy, in which the stent 1100 may be used to create an anastomosis between the stomach 1105 and small intestine 1107 to form a bypass of the duodenum. Details of the surgical procedure are described in U.S. Patent Application Publication No. 2019/0298401, which is herein incorporated by reference in its entirety.

In all of the above embodiments, the anti-migration features 150, 250, 350, 450, 550, 650, 750, 750, 950, 1050, 1150 may be formed by a single wire having opposing ends fixed to the tubular body to define a closed loop. The closed loop anti-migration features may be fixed to the tubular body such that any pulling or squeezing force applied to the anti-migration features does not result in a reduced diameter or elongation or shortening of the tubular body. As such, the anti-migration features are not intended to function as retrieval elements. Alternatively, the anti-migration features may be formed by a plurality of wire segments of a plurality of wires extending from the interwoven structure of the tubular body of the stent. In some instances, terminal ends of the plurality of wires are welded or otherwise secured together form the closed loop with a base end of the closed loop fixed to the tubular body. The base end of each closed loop may be located at a single cross-over point in the tubular body or the base end of each loop may be fixed to adjacent cross-over points. In all of the above described embodiments, the anti-migration features 150, 250, 350, 450, 550, 650, 750, 750, 950, 1050, 1150 may be moveable between a delivery configuration in which the anti-migration features extend substantially parallel to the central longitudinal axis of the tubular body of the stent, and a deployed configuration in which the anti-migration features extend radially away from the central longitudinal axis, where the anti-migration features are biased in the deployed configuration when unconstrained and/or the stent is deployed to the expanded configuration. The anti-migration features may be held in the delivery configuration by an outer sheath disposed over the stent. Releasing the stent from the outer sheath will allow the anti-migration features to expand to their angled configuration. In other embodiments, a suture or wire may be threaded through the anti-migration features to hold them in the delivery configuration. Upon delivery, the suture or wire is removed to allow the anti-migration features to return to their biased, angled configuration.

Any of the stents 200, 300, 400, 500, 600, 700, 800, 900, 1000, 1100 described above may include a covering 170 as described in relation to the stent 100 shown in FIG. 1.

It will be understood that any angles described in association with the above figures are illustrative only, and that other angles of the closed loop anti-migration features are contemplated. The materials that can be used for the various components of the stent 100, 200, 300, 400, 500, 600, 700, 800, 900, 1000, 1100 and the various elements thereof disclosed herein may include those commonly associated with medical devices. For simplicity purposes, the following discussion refers to the stent 100 (and variations, systems or components disclosed herein). However, this is not intended to limit the devices and methods described herein, as the discussion may be applied to other elements, members, components, or devices disclosed herein.

In some embodiments, the stent 100 (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super-elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super-elastic plateau and/or flag region that may be seen with super-elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the stent 100 (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent 100 (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent 100 (and variations, systems or components thereof disclosed herein) to achieve the same result.

In some embodiments, the stent 100 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the stent 100 (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent comprising:
a tubular body formed of interwoven wires, the tubular body having a first open end, an opposing second open end, and a central longitudinal axis extending therebetween, the tubular body moveable between a radially compressed state and a radially expanded state; and
a plurality of anti-migration features each having a first end positioned at an outer surface of the tubular body and a second end extending radially outward from the outer surface of the tubular body;
wherein each of the plurality of anti-migration features is defined by a closed loop of one or more of the interwoven wires with a base of the closed loop located at the outer surface of the tubular body.

2. The stent of claim 1, wherein the base of the closed loop includes a cross-over point of the one or more interwoven wires forming the closed loop.

3. The stent of claim 2, wherein the one or more interwoven wires are welded at the cross-over point.

4. The stent of claim 3, wherein any pulling or squeezing force applied to any of the plurality of anti-migration features does not reduce an outer diameter of the tubular body or axially lengthen or shorten the tubular body.

5. The stent of claim 2, wherein a first portion of the plurality of anti-migration features are coupled to the tubular body adjacent the first open end and extend towards the second open end at an acute angle relative to the outer surface of the tubular body.

6. The stent of claim 5, wherein a second portion of the plurality of anti-migration features is coupled to the tubular body adjacent the second open end and extend towards the first open end at an acute angle.

7. The stent of claim 1, wherein a first portion of the plurality of anti-migration features is coupled to a medial region of the tubular body and extend towards the first open end at an acute angle, and a second portion of the plurality of anti-migration features is coupled to the medial region of the tubular body and extend towards the second open end at an acute angle.

8. The stent of claim 7, wherein the base of each anti-migration feature of the first portion and the base of each anti-migration feature of the second portion are circumferentially spaced apart at a single longitudinal location along the tubular body.

9. The stent of claim 1, wherein the closed loops defining the plurality of anti-migration features are located at the first open end and extend radially outward from the tubular body.

10. The stent of claim 9, further comprising a plurality of elongated closed loops at the first open end extend substantially parallel to the central longitudinal axis.

11. The stent of claim 10, wherein the plurality of elongate closed loops is interposed between adjacent ones of the closed loops defining the plurality of anti-migrations features.

12. The stent of claim 1, wherein each closed loop is formed by a plurality of the interwoven wires, wherein terminal ends of the plurality of interwoven wires are welded around a periphery of the closed loop.

13. The stent of claim 12, wherein each closed loop is formed by segments of four of the interwoven wires collectively defining the periphery of the closed loop.

14. The stent of claim 12, wherein the base includes a cross-over point of first and second wires of the interwoven wires forming the closed loop.

15. The stent of claim 14, wherein the first and second wires are welded together at the cross-over point.

16. A stent comprising:
a tubular body formed of interwoven wires, the tubular body having a first open end, an opposing second open end, and a central longitudinal axis extending therebetween, the tubular body moveable between a radially compressed state and a radially expanded state; and
a plurality of anti-migration features each having a first end welded to one or more cross-over points of the one or more interwoven wires forming the tubular body, and a second end extending radially outward from an outer surface of the tubular body.

17. The stent of claim 16, wherein each of the plurality of anti-migration features is formed, at least in part, by a wire of the interwoven wires forming the tubular body.

18. The stent of claim 16, wherein each of the plurality of anti-migration features is formed by a plurality of wires of the interwoven wires arranged in a closed loop, wherein terminal ends of the plurality of wires are welded around a periphery of the closed loop.

19. A stent comprising:
a radially expandable tubular body formed of interwoven wires, the tubular body having a first open end, an opposing second open end, and a central longitudinal axis extending therebetween, the tubular body moveable between a radially compressed state and a radially expanded state; and
a plurality of anti-migration features located at the first open end, each of the plurality of anti-migration features having a first end positioned at an outer surface of the tubular body and a second end extending radially outward from the outer surface of the tubular body;
wherein each of the plurality of anti-migration features is formed by a plurality of wires of the interwoven wires arranged in a closed loop with terminal ends of the plurality of wires arranged around a periphery of the closed loop.

20. The stent of claim 19, wherein the terminal end of the plurality of wire are welded around the periphery of the closed loop.
